(19) United States
(12) Patent Application Publication
(10) Pub. No.: US 2021/0338053 A1
(43) Pub. Date: Nov. 4, 2021

(54) WIRE BIASING GUIDE FOR A MEDICAL DEVICE

(71) Applicant: Gyrus ACMI, Inc. d/b/a Olympus Surgical Technologies America, Westborough, MA (US)

(72) Inventors: Taylor N. Tyson, Seattle, WA (US); Peter A. Lambe, Redmond, WA (US); Michael S. Smith, Sammamish, WA (US)

(21) Appl. No.: 17/374,391

(22) Filed: Jul. 13, 2021

Related U.S. Application Data
(63) Continuation of application No. 15/994,033, filed on May 31, 2018, now abandoned.

(51) Int. Cl. A61B 1/005; A61B 1/00; A61B 1/012

(52) U.S. Cl. CPC A61B 1/0052; A61B 1/00066; A61B 1/0057; A61B 1/00071; A61B 1/012

(57) ABSTRACT

An apparatus for controlling positioning of wires and/or tubing that extends from a handle of a medical device. An exemplary apparatus includes a device attachment mechanism configured to removably attach to a handle of a medical device and a guide component configured to maintain a plurality of different positions relative to the device attachment mechanism and the handle of the medical device. The guide component allows a user to alter the direction (i.e., biases) any wires or tubing, such as electrical wires or saline tubes that extend from the handle to other instrumentation, such as pumps or generators.

### PRIORITY CLAIM

This application is a Continuation Application of U.S. patent application Ser. No. 15/994,033 filed May 31, 2018, which is hereby incorporated by reference.

### BACKGROUND

Currently many medical devices that transport fluid and/or electrical energy to components inside the patient's body include wires/tubing that connects to more stationary tower-based devices. These wires/tubing can be a nuisance to an operator who is manipulating the medical device. There only exists simple devices, such as zip ties, clips or holders that are used for managing wires and tubing.

### SUMMARY

Devices, systems, and methods are provided for controlling positioning of wires and/or tubing that extends from a handle of a medical device.

In one aspect, an illustrative a device includes a base section configured to mount to a proximal end of an endoscope and a wire biasing component couplable to the base section. The wire biasing component is configured to receive one or more wires extending from the proximal end of the endoscope through the base section. The wire biasing component is configured to bias the received wires in a direction different than an unbiased direction.

In another aspect, a system includes a medical device having one or more wires extending from a proximal end of an endoscope and a wire biasing device having a base section configured to mount to the proximal end of the endoscope and a wire biasing component couplable to the base section. The wire biasing component is configured to receive one or more wires from the endoscope through the base section. The wire biasing component is configured to bias the received wires in a direction different than an unbiased direction.

In yet another aspect, a method provides a base section configured to mount to a proximal end of an endoscope and provides a wire biasing component couplable to the base section. The wire biasing component is configured to receive one or more wires from the endoscope through the base section. The method biases the received wires in a direction different than an unbiased direction.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1 shows a medical device with a wire management device formed in accordance with an embodiment of the present invention;

FIG. 2 shows a system implementing the devices of FIG. 1;

FIG. 3 shows a perspective view of a wire management device formed in accordance with an embodiment of the present invention;

FIG. 4 is a perspective view of a distal end of a medical device implementing the wire management device shown in FIG. 3;

FIG. 5 shows a perspective view of a wire management device formed in accordance with an embodiment of the present invention;

FIG. 6 shows a side view of a wire management device similar to the wire management device shown in FIG. 5;

FIG. 7 shows a perspective view of a distal end of a medical device implementing the wire management device shown in FIG. 5;

FIG. 8 shows a perspective view of a wire management device formed in accordance with an embodiment of the present invention;

FIG. 9 shows a system implementing a wire management device formed in accordance with an embodiment of the present invention; and

FIG. 10 shows a cross-sectional view of the wire management device used with the system of FIG. 9.

### DETAILED DESCRIPTION

The following description is merely illustrative in nature and is not intended to limit the present disclosure, application, or uses. The following description explains, by way of illustration only and not of limitation, various embodiments of devices for biasing wire/tubing positions relative to a medical device, as well as systems including such devices and methods of using the same. It will be appreciated that various embodiments of biasing devices described herein may help to simplify the process of positioning and manipulating the medical devices.

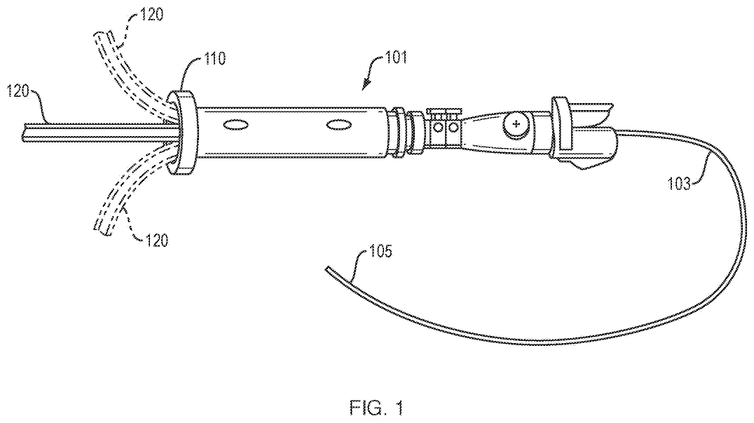

Referring to FIG. 1, a medical device 101 includes a handle that may include one or more movable components. The medical device 101 includes an insertion component 103 that extends from a distal end of the handle to a distal operative end 105 that is placed within a patient at or near target tissue. Wires and/or tubing (pneumatic or fluidic) 120 extend from a proximal end of the handle. The direction the wires and/or tubing 120 extends from the handle is controlled (i.e., biased) by a biasing device 110. Examples of different biasing devices 110 are shown and described below by example. The wires/tubing 120 may connect to various tower-based (remote) equipment, such as an electrosurgical radio frequency (RF) generator, an infusion pump, or a pneumatic pump, for controlling features or components, such as an electrosurgical instrument, a bipolar or monopolar RF device, etc. located at the distal operative end 105.

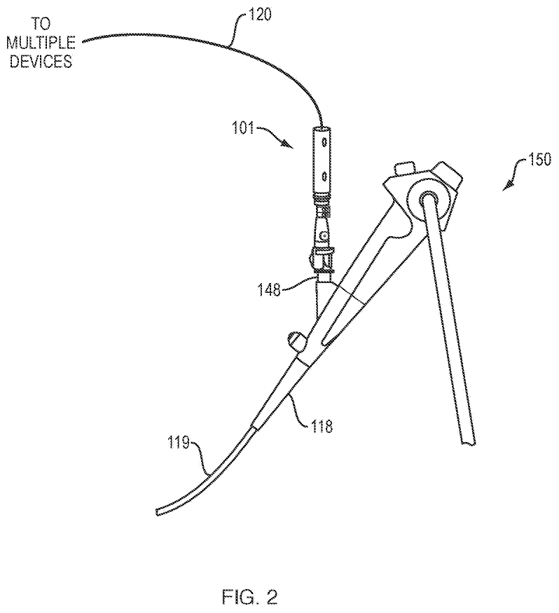

Referring to FIG. 2, the medical device 101 is shown being used in conjunction with a scope device 118, such as a bronchoscope, for treating, sampling or examining tissue at a reference point in an anatomical region of a patient (not shown).

In various embodiments, the scope device 118 includes an insertion tube 119 that permits insertion of the insertion component 103 into a body (not shown). The distal operative end 105 of the insertion component 103 is delivered to a location near the tissue to be treated at or near the reference point. Positioning of the distal operative end 105 of the insertion component 103 may be controlled by control features of the medical device 101 or the scope device 118. The insertion component 103 is received through a port 148 of the scope device 118.

The biasing device 110 allows for user to adjust positioning of the tubing and wires for a given procedure. The biasing device 110 permits the user to select where they want to the wires and/or tubing to be located, thus keeping them from getting in the way during a procedure. The biasing device 110 biases the wires and/or tubing to a desired side/position away from any sections of interest on the medical device 101 or from interfering with movement or positioning of the person operating the medical device 101.

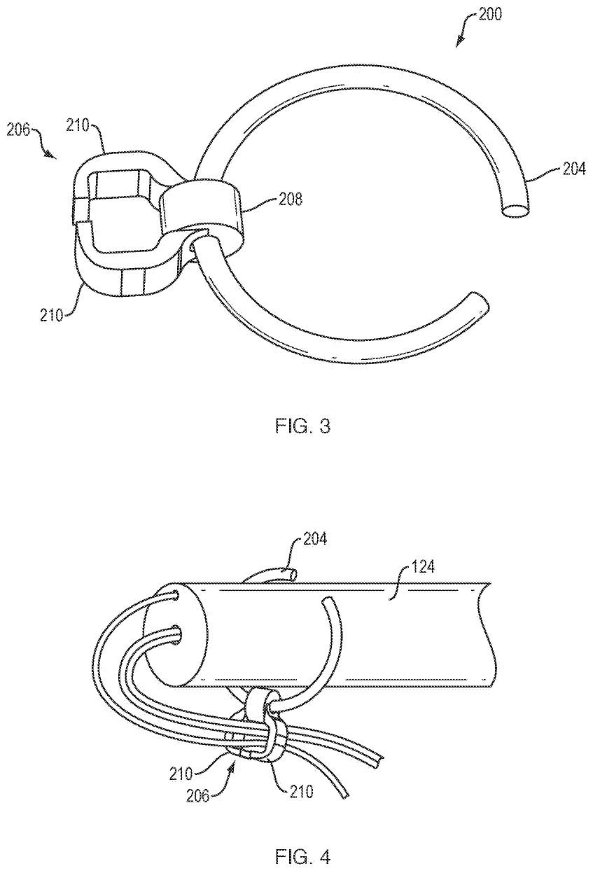

As shown in FIG. 3, an exemplary biasing device 200 includes a rotating holder 206 mounted to a ring 204. The rotating holder 206 directs the tubing and/or wires based on its position on the ring 204. The ring 204 is sized to hold on to a body section of a medical device 124 (FIG. 4), such as the medical device 101 (FIG. 1). A user would place the tubing and/or wires of the medical device 124 in the rotating holder 206 and slide the rotating holder 206 along the ring 204 to the desired location. In one embodiment, the medical device 120 includes a feature (not shown) for receiving and/or positioning the ring 204. In one embodiment, the ring 204 is an expandable rod that can be expanded to allow for insertion of a medical device handle.

The ring 206 includes a base section 208 and one or more support arms 210 that are attached to the base section 208. In one embodiment, the base section 208 includes a curved lumen for receiving the ring 204. The base section 208 maintains position on the ring 204 due to a fictional relationship, a ratcheting relationship or any relationship that allows the user to move the base section 208 about the ring 204, then once the user force is no longer applied, the base section 208 maintains its position on the ring 204.

FIG. 4 shows an example of wire and tubing that extends from a proximal surface of the medical device 124. The wires and tubing may exit the medical device 124 at other locations. The wires and tubing pass through the support arms 210, thus causing the wires and tubing to be directed in approximately a distal direction until they exit the support arms 210. Upon exiting the support arms 210, the wires and tubing bend towards their associated instruments.

The support arms 210 may be replaced by various other types of support mechanism, for example a carabiner type support device may be used.

In one embodiment, as shown in FIG. 5, a biasing device 300 includes a rotating disk 302 with one or more holes 310 and a support mount 301. The support mount 301 holds the rotating disk 300 in place and allows for the rotating disk 300 to rotate. The tubing or wire is placed one or more of the holes 310. A tab 312 extends from the rotating disk 302 outside outer edges of the support mount 301. The support mount 301 includes top and bottom halves 304, 306 that connected via a central hub 330. The central hub 330 is received through a center hole 334 of the rotating disk 302. The inner diameter of rotating disk 302 is slightly larger than the outer diameter of the central hub 330, thus allowing the rotating disk 302 to rotate about the central hub 330. The user can rotate the tab 312 and thus the disk 302 to move the wire and tubing to a desired side of the medical device in which the biasing device 300 is attached to ensure the wires and tubing are out of the way of the user. In one embodiment, the support mount 301 only includes one side with a hub for receiving the disk.

In one embodiment, an arm 320 of at least one of the halves 304, 306 connects a center portion to an outer portion. The arm 320 includes a ball portion 332 of a ball catch device. The rotating disk 302 includes one or more ball for receiving dimples 314 that are located the same radial distance from a center of the central hub 330 as the ball portion 332. The size of the ball portion 332 and the ball receiving dimples 314 are selected so that force applied to the tab 312 causes the ball portion 332 to unseat from the ball receiving dimple 314. Without the force on the tab 312, the ball portion 332 remains seated within the ball receiving dimple 314.

Other types of motion restriction device may be used so that the components may stay in place until actively acted upon by a user. For example, the ball portion 332 may be a ball spring plunger on one component with or without the use of a dimple on the other component. Also, the components may be sized such that a friction fit occurs when the arm 320 overlaps the rotating disk 302.

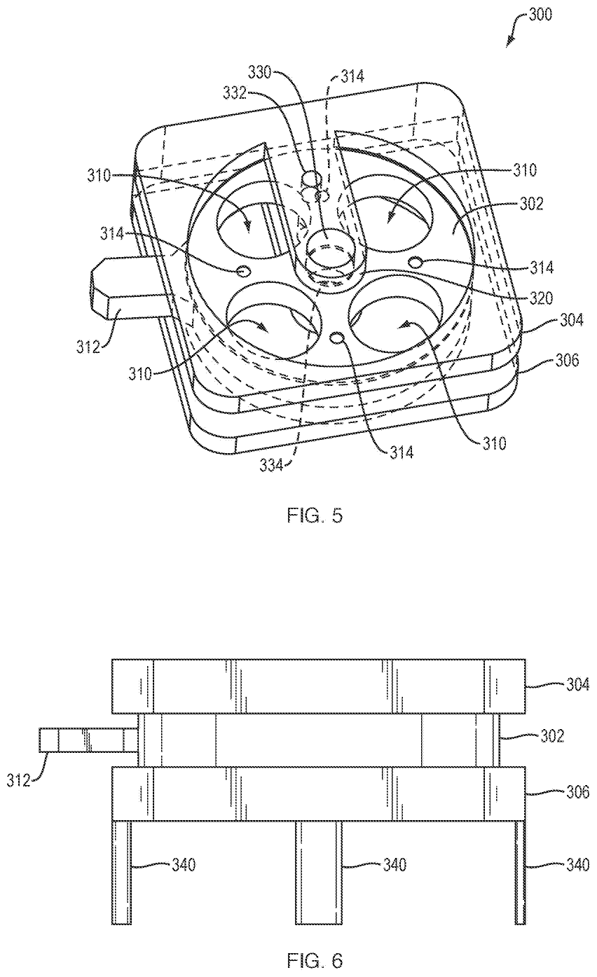

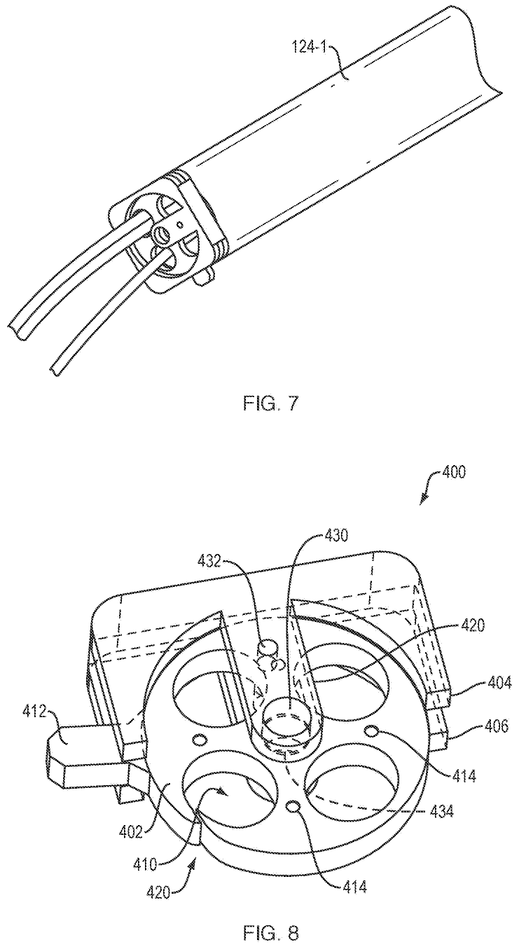

FIG. 6 shows a side view of an embodiment of the biasing device 300. A plurality of gripping arms 340 extend from a distal end of one of the halves 306. The gripping arms 340 are spaced in order to grip onto a medical device 124-1 (FIG. 7). The inner surface of the gripping arms 340 may include a gripping pad (e.g., rubber pads) (not shown) for gripping onto the medical device 124-1. In one embodiment, the medical device 124-1 includes a feature (not shown) for receiving the gripping arms 340 and/or for receiving one or more of the halves 304, 306 or features included in the halves 304, 306.

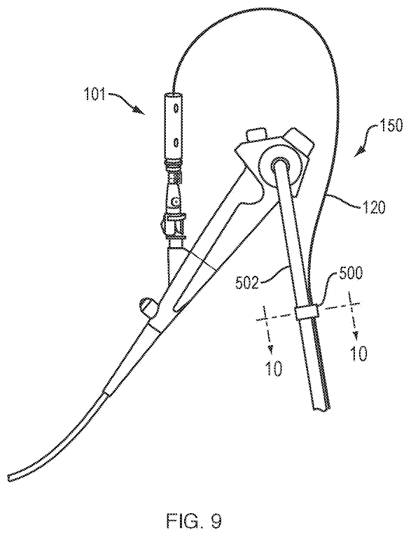

FIG. 8 shows a perspective view of a biasing device 400 having an open configuration in contrast to that shown in FIG. 5. The biasing device 400 is similar to the biasing device 300 of FIGS. 5-7 in that the biasing device 400 includes mount halves 404, 406 and a rotating disk 402 with a tab 412 and one or more holes 410. However, the mount halves 404, 406 do not extend fully around the rotating disk 402. Also at least one of the holes 410 on the rotating disk 402 includes a gap or an opening latch 420, similar to that may be seen on a carabiner. Other gating/latching devices may be used for the opening latch 420. The wires and/or tubing are inserted into the hole 410 via the latch 420. The biasing device 400 may mount to the medical device 124-1 in a manner similar to those described above.

Referring to FIG. 9, the medical device 101 is shown being used in conjunction with a scope device, such as a bronchoscope, for treating, sampling or examining tissue at a reference point in an anatomical region of a patient (not shown). The scope device includes a data/signal cable for communicating with tower-based equipment. A wire control device 500 allows for user to temporarily clip tubing and wires of the medical device 101 to the scope cable. The wire control device 500 keeps the cables, tubing and wires all in one area, thus keeping them from getting in the way during a procedure.

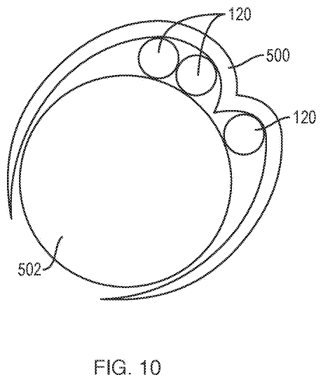

As shown in FIG. 10, the wire control device 500 is a w-shaped expandable clip that holds the tubing and wires 120 of the medical device 101 to the cable 502 of the scope device. The wire control device 500 includes two interior curved base sections. When the wire control device 500 receives the cable 502, a first passage exists between the cable 502 and the first interior curved base section and a second passage exists between the cable 502 and the second interior curved base section. The first passage may receive electrical wires associated with the electrodes of the medical device 101. The second passage may receive a saline tube that extends from the medical device 101. The wire control device 500 may be made from any number of flexible materials (e.g. plastic) that can be flexed wide enough to receive the tubing and wires 120 and the cable 502 and have a clamping strength strong enough to hold the tubing and wires 120 to the cable 502.

### Embodiments

A. A device comprising: a base section configured to mount to a proximal end of an endoscope; and a wire biasing component couplable to the base section, the wire biasing component is configured to receive one or more wires extending from the proximal end of the endoscope through the base section, wherein the wire biasing component is configured to bias the received wires in a direction different than an unbiased direction.

B. The device of A, wherein the wire biasing component includes a first latch configured to allow access to a first one of the wires while restricting exit of the first wire from the wire biasing component.

C. The device of A or B, wherein the wire biasing component comprises a first passageway configured to apply a biasing force to a first one of the wires.

D. The device of C, further comprising a position stabilizing device configured to resist a force configured to counter the biasing force.

E. The device of B, wherein the wire biasing component includes a second latch configured to allow access to a second one of the wires while restricting exit of the second wire from the wire biasing component.

F. The device of C, wherein the wire biasing component comprises a second passageway configured to apply a biasing force to a second one of the wires.

G. The device of F, further comprising a position stabilizing device configured to resist a force configured to counter the biasing force.

H. A system comprising: a medical device comprising one or more wires extending from a proximal end of an endoscope; and a wire biasing device comprising: a base section configured to mount to the proximal end of the endoscope; and a wire biasing component couplable to the base section, the wire biasing component is configured to receive one or more wires from the endoscope through the base section, wherein the wire biasing component is configured to bias the received wires in a direction different than an unbiased direction.

I. The system of H, wherein the wire biasing component includes a first latch configured to allow access to a first one of the wires while restricting exit of the first wire from the wire biasing component.

J. The system of H or I, wherein the wire biasing component comprises a first passageway configured to apply a biasing force to a first one of the wires.

K. The system of J, wherein the wire biasing device further comprises a position stabilizing device configured to resist a force configured to counter the biasing force.

L. The system of I, wherein the wire biasing component includes a second latch configured to allow access to a second one of the wires while restricting exit of the second wire from the wire biasing component.

M. The system of J, wherein the wire biasing component comprises a second passageway configured to apply a biasing force to a second one of the wires.

N. The system of M, the wire biasing device further comprises a position stabilizing device configured to resist a force configured to counter the biasing force.

O. A method comprising: providing a base section configured to mount to a proximal end of an endoscope; providing a wire biasing component couplable to the base section, the wire biasing component is configured to receive one or more wires from the endoscope through the base section; and biasing the received wires in a direction different than an unbiased direction.

P. The method of O, further comprising: latching a first one of the wires within the wire biasing component.

Q. The method of O or P, wherein biasing comprises: applying a biasing force to the first one of the wires.

R. The method of P, further comprising: latching a second one of the wires within the wire biasing component.

S. The method of R, wherein biasing comprises: applying a biasing force to the second one of the wires.

T. The method of O, further comprising: resisting a force configured to counter the biasing force.

The description of the invention is merely exemplary in nature and variations that do not depart from the gist of the invention are intended to be within the scope of the invention. Such variations are not to be regarded as a departure from the spirit and scope of the invention.

Although the preferable embodiments of the present invention have been described hitherto, the present invention is not limited to these embodiments. Additions, omissions, substitutions, and other modifications can be made without departing from the spirit or scope of the present invention.

In addition, the invention is not limited by the above description and is limited by only the scope of appended claims.

What is claimed is:

1. A device comprising:
a base section configured to mount to a proximal end of an endoscope; and
a wire biasing component couplable to the base section, the wire biasing component is configured to receive one or more wires extending from the proximal end of the endoscope through the base section,
wherein the wire biasing component is configured to bias the received wires in a direction different than an unbiased direction.

2. The device of claim 1, wherein the wire biasing component includes a first latch configured to allow access to a first one of the wires while restricting exit of the first wire from the wire biasing component.

3. The device of claim 1, wherein the wire biasing component comprises a first passageway configured to apply a biasing force to a first one of the wires.

4. The device of claim 3, further comprising a position stabilizing device configured to resist a force configured to counter the biasing force.

5. The device of claim 2, wherein the wire biasing component includes a second latch configured to allow access to a second one of the wires while restricting exit of the second wire from the wire biasing component.

6. The device of claim 3, wherein the wire biasing component comprises a second passageway configured to apply a biasing force to a second one of the wires.

7. The device of claim 6, further comprising a position stabilizing device configured to resist a force configured to counter the biasing force.

8. A system comprising:
a medical device comprising one or more wires extending from a proximal end of an endoscope; and
a wire biasing device comprising:
a base section configured to mount to the proximal end of the endoscope; and
a wire biasing component couplable to the base section, the wire biasing component is configured to receive one or more wires from the endoscope through the base section,
wherein the wire biasing component is configured to bias the received wires in a direction different than an unbiased direction.

9. The system of claim 8, wherein the wire biasing component includes a first latch configured to allow access to a first one of the wires while restricting exit of the first wire from the wire biasing component.

10. The system of claim 8, wherein the wire biasing component comprises a first passageway configured to apply a biasing force to a first one of the wires.

11. The system of claim 10, wherein the wire biasing device further comprises a position stabilizing device configured to resist a force configured to counter the biasing force.

12. The system of claim 9, wherein the wire biasing component includes a second latch configured to allow access to a second one of the wires while restricting exit of the second wire from the wire biasing component.

13. The system of claim 10, wherein the wire biasing component comprises a second passageway configured to apply a biasing force to a second one of the wires.

14. The system of claim 13, the wire biasing device further comprises a position stabilizing device configured to resist a force configured to counter the biasing force.

15. A method comprising:
providing a base section configured to mount to a proximal end of an endoscope;
providing a wire biasing component couplable to the base section, the wire biasing component is configured to receive one or more wires from the endoscope through the base section; and
biasing the received wires in a direction different than an unbiased direction.

16. The method of claim 15, further comprising:
latching a first one of the wires within the wire biasing component.

17. The method of claim 16, wherein biasing comprises:
applying a biasing force to the first one of the wires.

18. The method of claim 16, further comprising:
latching a second one of the wires within the wire biasing component.

19. The method of claim 18, wherein biasing comprises:
applying a biasing force to the second one of the wires.

20. The method of claim 15, further comprising:
resisting a force configured to counter the biasing force.
